(19) United States
(12) Patent Application Publication
(10) Pub. No.: US 2019/0350980 A1
(43) Pub. Date: Nov. 21, 2019

(54) COMPOSITIONS AND METHODS FOR TRANSPLANT RECIPIENT CONDITIONING

(71) Applicants: Thalia PAPAYANNOPOULOU, Seattle, WA (US); Halvard B. BONIG, Seattle, WA (US); Michael HUDECEK, Seattle, WA (US)

(72) Inventors: Thalia PAPAYANNOPOULOU, Seattle, WA (US); Halvard B. BONIG, Seattle, WA (US); Michael HUDECEK, Seattle, WA (US)

(73) Assignee: DRK BLUTSPENDEDIENST BADEN-W?RTTEMBERG-HESSEN GMBH, Frankfurt Am Main (DE)

(21) Appl. No.: 16/478,995

(22) PCT Filed: Jan. 18, 2018
(86) PCT No.: PCT/US2018/014186

§ 371 Date: Jul. 18, 2019

Related U.S. Application Data
(60) Provisional application No. 62/447,620, filed on Jan. 18, 2017.

(51) Int. Cl. A61K 35/17; A61K 38/17

(52) U.S. Cl. CPC A61K 35/17; A61K 45/06; A61K 38/1774

(57) ABSTRACT

Described herein are methods of promoting engraftment of a cell transplant and methods of killing hematopoietic stem cells by administering a population of T cells, NK cells or cytotoxic immune effector cells comprising a cell-surface receptor for a stem cell-specific antigen. Also described herein is a composition comprising T cells, NK cells or cytotoxic immune effector cells genetically modified to encode a cell-surface receptor for a stem cell-specific antigen.

### CROSS-REFERENCE TO RELATED APPLICATIONS

This application claims benefit under 35 U.S.C. § 119(e) of U.S. provisional application 62/447,620 filed Jan. 18, 2017, the content of which is incorporated herein by reference in its entirety.

### FIELD OF THE INVENTION

The invention relates to the promotion of transplant engraftment.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Recipients of allogeneic hematopoietic stem cell transplants must receive a so-called “conditioning” prior to graft transfusion, which ablates host hematopoiesis, so that donor hematopoiesis can engraft. Conditioning regimens for allogeneic transplantation are typically myeloablative and lymphoablative in order to eliminate malignant cells (to eradicate disease) and recipient lymphoid cells (to allow durable donor hematopoietic stem-cell engraftment; to eliminate graft-reactive host cells). Typically this entails a high-dose chemotherapy, sometimes in combination with whole-body irradiation and/or serotherapy with immune cell-depleting antibodies, possessing very high stem cell and immune cell toxicity. Such regimens are associated with substantial risk of morbidity and mortality. The lack of specificity of all these components (except for the serotherapy) for hematopoietic cells explains the very high off-target morbidity and mortality to conditioning associated toxicity. While the chemo-/radio-conditioning may be required for patients with underlying malignant diseases, these effects are undesirable in patients with non-malignant diseases, such as aplastic anemias, primary immunodeficiencies, hemoglobinopathies; and are intolerable in patients (currently precluded from transplantation as a therapeutic option) with defects in DNA repair who are prone to hematopoietic malignancies, such as ataxia telangiectasia, Bloom syndrome, and Fanconi anemia.

Older patients (age >50 years) and patients with confounding medical conditions are frequently not eligible for allogeneic transplantation because of the risks associated with these regimens. Regimen-related toxicity is a considerable cause of morbidity and mortality in transplant patients and can include low blood counts, anemia, neutropenia, thrombocytopenia, fatigue, infection, fever, mouth sores, nausea and vomiting, hair loss, pain, depression, and reproductive and sexual dysfunction. Nausea, vomiting, stomatitis, enteritis, alopecia, erythema or rash, and diarrhea occur in most graft recipients and can largely be controlled. More serious complications might include idiopathic interstitial pneumonitis, hemorrhagic cystitis, heart failure and/or pericarditis, hepatic veno-occlusive disease (VOD), and, less commonly, pulmonary hemorrhage.

However, there remains a need in the art for methods of promoting engraftment of a transplant e.g., a hematopoietic stem cell (HSC) transplant, and circumventing the toxicity to other cells or tissues when conditioning the subjects for a cell transplant.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 depicts in accordance with various embodiments of the invention, dose dependent specific cytotoxicity of healthy donor bone marrow, fresh, 20 h co-culture with NK92 cells. Bars (from left to right) represent Colony Forming Unit-Culture (CFU-C) growth of equal numbers of bone marrow cells coincubated with: no NK92 cells (bar 1), anti-c-kit CAR SR1-NK92 cells at t:e ratios of 30:1, 10:1, 3:1 (bars 2-4). This is one representative experiment of five similar experiments. Within each bar, from top to bottom, are depicted granulocyte-monocyte (GM), monocyte (M), granulocyte (G), and erythroid (E) populations.

FIG. 2 depicts in accordance with various embodiments of the invention, dose dependent specific cytotoxic effects of an anti-mouse CAR directed against murine c-kit. The target population is murine (C57Bl/6) bone marrow. The effect is similar to the effect of the anti-human c-kit CAR against human bone marrow. Within each bar in the left side graph, from top to bottom, are depicted granulocyte-monocyte (GM), monocyte (M), granulocyte (G), and erythroid (E) populations.

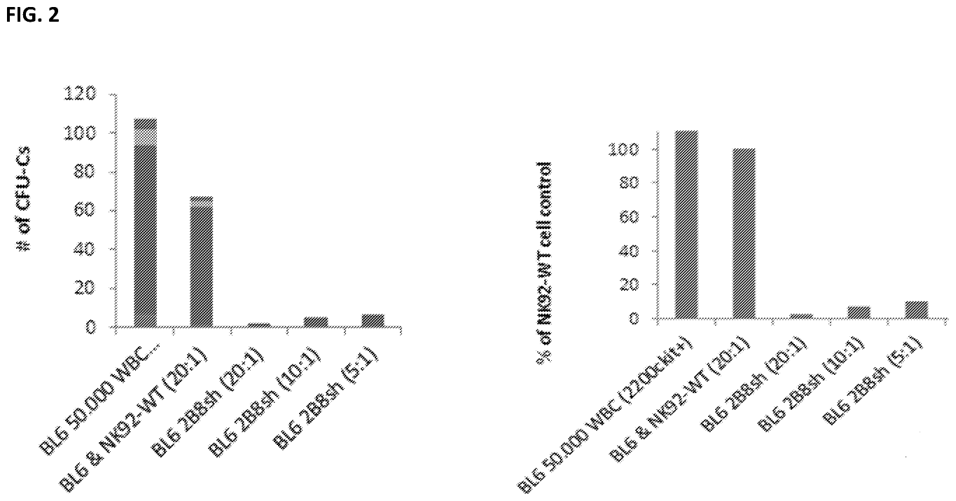

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As described herein, “donor cells” can refer to donor T lymphocytes, donor bone marrow cells and other immune cells used in a transplant setting. Donor cells can be allogeneic or autologous.

“Cell surface antigen”, as described herein refers to a protein expressed on the surface of a cell that can be used as a target for an immune cell or immune response.

“Hematopoietic specific antigen”, as described herein refers to a protein, carbohydrate, or glycoprotein that is expressed predominantly on the surface of a hematopoietic cell. Antigens expressed predominantly on the surface of a stem cell include antigens expressed solely on the surface of a stem cell as well as antigens expressed on other cells to a much lesser degree, i.e., at least 5 fold less abundantly on non-stem cells, at least 10-fold less abundantly, at least 20-fold less abundantly or below. Different types of stem cells express different cell surface markers and therefore the specific type of stem cell can be identified by the presence of selected cell surface markers. Examples of hematopoietic stem cell-specific antigens include, but are not limited to c-kit (CD117), CD33, CD123 (IL-3Ralpha), CD133, CD135, CD105 and CD45.

“Stem cells”, as referred to herein are pluripotent or multipotent cells that can differentiate into multiple cell types. In various embodiments, the cells are hematopoietic stem cells. A “precursor cell” or “progenitor cell” can be any cell in a specific differentiation pathway that is capable of differentiating into a more mature cell and can comprise cells that are totipotent, cells that are pluripotent and cells that are stem cell lineage restricted (i.e., cells capable of developing into less than all hematopoietic lineages). As used herein, the term “totipotent cell” refers to a cell capable of developing into all lineages of cells. Also as used herein, the term “pluripotent cell” refers to a cell capable of developing into a variety of (albeit not all) lineages and are at least able to develop into all hematopoietic lineages (e.g., lymphoid, erythroid, and thrombocytic lineages). As used herein, the terms “develop” and “differentiate” refer to the progression of a cell from the stage of having the potential to differentiate into at least two different cellular lineages to becoming a specialized cell.

The term “protein”, as used herein, includes proteins, polypeptides and peptides and refers to polymers of amino acids of any length and may be an intact molecule, a fragment thereof, or multimers or aggregates of intact molecules and/or fragments; and may occur in nature or be produced, e.g., by synthesis (including chemical and/or enzymatic) or genetic engineering. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (for example, unnatural amino acids, etc.), as well as other modifications known in the art.

The term “nucleic acid” or “polynucleotide”, as used herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as, but not limited to methylated nucleotides and their analogs or non-nucleotide components. Modifications to the nucleotide structure may be imparted before or after assembly of the polymer. A polynucleotide may be further modified after polymerization, such as by conjugation with a labeling component.

The term “stem cell transplantation” as used herein includes infusion into a patient of stem cells, including but not limited to hematopoietic stem cells, derived from any appropriate source of stem cells in the body. In certain embodiments, the stem cells may be derived, for example, from bone marrow, from the peripheral circulation following mobilization from the bone marrow, or from fetal sources such as fetal tissue, fetal circulation and umbilical cord blood. “Hematopoietic stem cell transplantation” as used herein, refers to the transplantation of multi-potent hematopoietic stem cells, usually derived from bone marrow, peripheral blood, or umbilical cord blood. It can be autologous, allogeneic or syngeneic.

As used herein, a “T cell”, may also be referred to as a T lymphocyte. It is a type of lymphocyte (a subtype of white blood cell) that plays a central role in cell-mediated immunity. T cells can be distinguished from other lymphocytes, such as B cells and natural killer cells, by the presence of a T-cell receptor on the cell surface. T cells include, but are not limited to effector cells, helper cells, cytotoxic (killer) cells, memory cells and/or regulatory (suppressor) cells.

As used herein, “NK” refers to natural killer cells (also known as cytotoxic NK cells, K cells and killer cells). These cells are a type of lymphocyte (a white blood cell), a component of the innate immune system and play a major role in the host-rejection of both tumors and virally infected cells.

As used herein, “CAR” means chimeric antigen receptor.

As used herein, “TCR” means “T-cell receptor”.

Described herein are methods of promoting engraftment of an HSC transplant and methods of killing hematopoietic stem cells by administering a population of T cells, NK cells or cytotoxic immune effector cells comprising a cell-surface receptor for a stem cell-specific antigen; and a composition comprising a T cell, NK cell or cytotoxic immune effector cell genetically modified to encode a cell-surface receptor for a stem cell-specific antigen.

Recipients of allogeneic stem cell transplants must receive a so-called “conditioning” prior to graft transfusion, which ablates host stem cells so that donor stem cells can engraft. It should be understood that the approaches described herein are applicable to stem cell transplants, but are exemplified and discussed herein in terms of hematopoietic stem cell transplantation. Typically, conventional conditions for transplantation entail high-dose chemotherapy, sometimes in combination with whole-body irradiation and/or serotherapy with immune cell-depleting antibodies, possessing very high stem cell and immune cell toxicity. The lack of specificity of all these components (except for the serotherapy) for hematopoietic cells explains the very high off-target morbidity and mortality to conditioning associated toxicity. While the chemo-/radio-conditioning may be required for patients with underlying malignant diseases, these effects are undesirable in patients with non-malignant diseases, such as aplastic anemias, primary immunodeficiencies, hemoglobinopathies; and are intolerable in patients (currently precluded from transplantation as a therapeutic option) with defects in DNA repair who are prone to hematopoietic malignancies, such as ataxia telangiectasia, Bloom syndrome, and Fanconi anemia.

Similarly, experimental transplantation in mammals requires conditioning, typically involving the same components as in patients, causing morbidity, sometimes dose-limiting, to the recipients. Specifically recipients of xenotransplants, critical scientific tools for the study of human hematopoietic stem cells, do not tolerate a full conditioning. Methods promoting engraftment while circumventing off-target toxicity can provide a relevant contribution to laboratory animal welfare.

The methods described herein harness the potential of “killer” immune cells and targets them against stem cell antigens to ablate stem cells with maximal specificity and minimal off-organ toxicity. Without being bound to any particular theory, an advantage of the approach described herein is avoidance of toxicity to non-stem cell compartments (e.g., cells other than hematopoietic stem cells) and organs, such as gastro-intestinal tract, liver, skin, central nervous system, kidneys, bone marrow stroma, and thus avoidance of complications associated with this, such as liver and kidney failure and failure of barrier function of skin and mucosae.

As described herein, the inventors have genetically modified immune cells to target them against specific cell antigens to ablate host cells with maximal specificity and minimal off-organ toxicity, allowing the engraftment of a stem cell graft.

The present invention is based, at least in part, on these findings. Embodiments address the need in the art for methods of promoting engraftment of a cell transplant and methods of killing, for example, hematopoietic stem cells prior to an hematopoietic stem cell transplant, by administering a population of T cells, NK cells or cytotoxic immune effector cells comprising a cell-surface receptor for a stem cell-specific antigen. Embodiments further provide for a composition comprising T cells, NK cells or cytotoxic immune effector cells genetically modified to encode a cell-surface receptor for a stem cell-specific antigen.

### Methods of Promoting Engraftment

Various embodiments of the present invention provide for a method of promoting engraftment of a hematopoietic stem cell transplant, the method comprising administering, to a subject in need of a hematopoietic stem cell transplant, a composition comprising a population of T cells, NK cells or cytotoxic immune effector cells comprising a cell-surface receptor for a hematopoietic stem cell-specific antigen, the amount effective to ablate the host stem cells with the hematopoietic cell-specific antigen in the subject. In various embodiments, the T cells are cytotoxic T cells. In some embodiments, the population of T cells, NK cells or cytotoxic immune effector cells is autologous to the subject. In some other embodiments, the population of cells is allogeneic to the subject. In various embodiments, the population of cells comprise a heterologous nucleic acid encoding the cell-surface receptor. In various embodiments, the cell surface receptor comprises a chimeric antigen receptor (CAR). In various other embodiments, the cell surface receptor comprises a T cell receptor (TCR). In various embodiments, the cell surface receptor specifically binds a hematopoietic cell-specific antigen selected from the group consisting of c-kit (CD117), CD33, CD123 (IL-3Ralpha), CD133, CD135, CD105 and CD45.

In various embodiments, the population of T cells, NK cells or cytotoxic immune effector cells is administered systemically. In some embodiments, the population of cells is administered locally. In various other embodiments, the population of cells is administered intravenously, intraarterially, subcutaneously, or intraosseously.

In various other embodiments, a population of immune effector cells is administered to the subject. In some embodiments, the immune effector cells are genetically modified to target a cell-surface receptor. In yet other embodiments, the cell-surface receptor targeted is a stem cell-specific antigen.

In various embodiments, the method further comprises administering an effective amount of one or more further agents or treatments that are used in conventional conditioning. In some embodiments, the agent or treatment is selected from serotherapy, total body irradiation, chemotherapy (immune-depleting, myeloablative or stem-cell depleting), anti-lymphocyte globulin, anti-T-cell antibodies and/or immune-cell-depleting antibody treatment.

In various embodiments, the method further comprises administering donor stem cells to the subject. In some embodiments, autologous cells are administered to the subject.

In various embodiments, the method further comprises ablating the administered cells to avoid proliferation and on-target toxicity against the donor stem cells by administering an agent and/or drug or a treatment in an amount sufficient to kill the administered population of T cells, NK cells or cytotoxic immune effector cells. In some embodiments, the population of T cells, NK cells or cytotoxic immune effector cells comprises one or more heterologous nucleic acids encoding one or more polypeptides that induces death in the administered population of T cells, NK cells or cytotoxic immune effector cells using a selected agent and/or drug. It is also contemplated that the administrated population of cells comprises one or more heterologous nucleic acids/proteins that render the cells dependent upon the presence of an agent for survival, such that the cells can be selectively killed by stopping the administration of the agent, drug and/or treatment. In other embodiments, administering the agent, drug and/or treatment induces apoptosis of the administered population of T cells, NK cells or cytotoxic immune effector cells. In various embodiments, the administered population of T cells, NK cells or cytotoxic immune effector cells eradicates the subject's hematopoietic stem cells.

Various embodiments provide for promoting engraftment of a cell transplant. Various types of cell transplant include, but are not limited to hematopoietic stem cell transplant, bone marrow transplant, a CD34+ cell transplant and/or a purified stem cell transplant. In various embodiments, the recipient of the transplant is a mammal. In various other embodiments, the recipient of the transplant is a human.

In a further embodiment, the transplant is a xenogeneic transplant such as a transplant derived from a non-human primate. In various other embodiments, the transplant is derived from a human. In other embodiments, the transplant recipient is a mammal. In yet other embodiments, the transplant recipient is a mouse. In a further embodiment, the cells target host cells that cause rejection. In one embodiment, the cells are administered at the same time as the transplant. In yet other embodiments, cells are administered within 24 hours of the transplant, within 2 days of the transplant, within 3 days of the transplant, within 4 days of the transplant, within 5 days of the transplant, within 6 days of the transplant, or within 7 days of the transplant. In various embodiments, the cells administered are genetically modified. In various additional embodiments, the transplant patient is given no post-transplant immunosuppression.

In an additional embodiment, the methods described herein can be used in a setting where the transplant patient is receiving a transplant for the treatment of a malignant or a non-malignant disease. Malignant diseases include, but are not limited to, non-Hodgkin's lymphoma, chronic myelogenous leukemia (CML), and chronic lymphocytic leukemia (CLL), multiple myeloma, acute myelogenous leukemia, acute lymphoblastic leukemia, and other cancers. Non-malignant diseases include, but are not limited to, hematologic failure (such as, aplastic anemias, beta thalassemia and sickle cell amenia), hemoglobinopathies, an autoimmune disease, an immunodeficiency, or a congenital disorder. Thus, the T or NK cells of the present invention can be used to promote engraftment in any setting where an HSC transplant may be used.

Various embodiments of the technology described herein also provide for a method of killing hematopoietic stem cells in a subject in need of an hematopoietic stem cell transplant, the method comprising administering, to a subject in need of hematopoietic stem cell transplantation, a composition comprising a population of T cells, NK cells or cytotoxic immune effector cells comprising a cell-surface receptor for an hematopoietic stem cell-specific antigen.

In some embodiments, the population of T cells, NK cells or cytotoxic immune effector cells is autologous to the subject. In some other embodiments, the population of cells is allogeneic to the subject. In various embodiments, the population of cells comprises a heterologous nucleic acid encoding the cell-surface receptor. In various embodiments, the cell surface receptor comprises a chimeric antigen receptor (CAR). In various other embodiments, the cell surface receptor comprises a T cell receptor (TCR). In various embodiments, the cell surface receptor specifically binds a hematopoietic cell-specific antigen selected from the group consisting of c-kit (CD117), CD33, CD123 (IL-3Ralpha), CD133, CD135 CD105 and CD45.

In various embodiments, the method further comprises administering an effective amount of one or more agents or treatments used in conventional conditioning to kill stem cells, (e.g., hematopoietic stem cells). In some embodiments, the agent or treatment is selected from serotherapy, total body irradiation, chemotherapy (immune-depleting, myeoablative or stem-cell depleting), anti-lymphocyte globulin, anti-T-cell antibodies and/or immune-cell-depleting antibody treatment.

In various embodiments, the method further comprises administering donor hematopoietic stem cells to the subject. In some embodiments, autologous cells are administered to the subject.

In various embodiments, the method further comprises ablating the administered T cells, NK cells or cytotoxic immune effector cells to avoid proliferation and on-target toxicity against the donor stem cells by administering an agent and/or drug or a treatment in an amount sufficient to kill the administered population of cells. In various other embodiments, the population of cells comprises one or more heterologous nucleic acids encoding one or more polypeptides that induce death in the administered population of cells using a selected inducing agent and/or drug. In yet other embodiments, administering the agent, drug and/or treatment induces apoptosis of the administered population of cells. In some other embodiments, the administered population of cells eradicates the subject's hematopoietic stem cells.

In various embodiments, the T cells, NK cells or cytotoxic immune effector cells of the invention are primary T or NK cells or are T or NK cell lines. T cells include, but are not limited to effector cells, helper cells, cytotoxic (killer) cells, memory cells and/or regulatory (suppressor) cells. Such cells can be obtained from a number of sources, including peripheral blood mononuclear cells, bone marrow, cord blood, thymus, tissue biopsy, lymph node tissue, spleen tissue, or any other lymphoid tissue. Such cells can also be obtained from a xenogeneic source, for example, from mouse, rat, non-human primate, and pig. In certain embodiments, bone marrow NK cells are used.

In various embodiments, T cells, NK cells or cytotoxic immune effector cells can be genetically modified to introduce one or more polynucleotides encoding one or more proteins or chimeric proteins that regulate T, NK or immune effector cell function (e.g., cell surface receptors and/or cytokine receptors, specific T cell receptors (e.g., receptors that recognize host cells)), chimeric antigen receptors, chemokine receptors, adhesion molecules, homing receptors, and the like). In further embodiments, T cells, NK cells or cytotoxic immune effector cells can be genetically modified to target a hematopoietic stem cell-specific surface antigen, including, but not limited to c-kit (CD117), CD33, CD123 (IL-3Ralpha), CD133, CD135 CD105 and CD45.

The administered cells can be transfected using numerous RNA or DNA expression vectors known to those of ordinary skill in the art. Genetic modification can comprise RNA or DNA transfection using any number of techniques known in the art, for example electroporation (using e.g., the Gene Pulser II, BioRad, Richmond, Calif.), various cationic lipids, (LIPOFECTAMINE™, Life Technologies, Carlsbad, Calif.), or other techniques such as calcium phosphate transfection as described in Current Protocols in Molecular Biology, John Wiley & Sons, New York. N.Y. For example, 5-50 μg of RNA or DNA in 500 μl of Opti-MEM can be mixed with a cationic lipid at a concentration of 10 to 100 μg, and incubated at room temperature for 20 to 30 minutes. Other suitable lipids include LIPOFECTIN™ and LIPOFECTAMINE™. The resulting nucleic acid-lipid complex is then added to 1-3×10⁶ cells, preferably 2×10⁶, antigen-presenting cells in a total volume of approximately 2 ml (e.g., in Opti-MEM), and incubated at 37° C. for 2 to 4 hours. The administered cells can also be transduced using viral transduction methodologies as described below.

The T cells, NK cells or cytotoxic immune effector cells may alternatively be genetically modified using retroviral or lentiviral transduction technologies. In various embodiments, the vector may be an amphotropic retroviral vector, preferably a vector characterized in that it has a long terminal repeat sequence (LTR)₅ e.g., a retroviral vector derived from the Moloney murine leukemia virus (MoMLV), human immunodeficiency virus (HIV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. Retroviruses adaptable for use in accordance with the present invention can be derived from any avian or mammalian cell source.

Various embodiments of the technology described herein provide for T cells, NK cells or cytotoxic immune effector cells that are genetically modified to express or overexpress a CAR or TCR that targets a surface antigen on hematopoietic stem cells. In various embodiments, the surface antigen is a hematopoietic specific target molecule. In various other embodiments, the surface antigen is a hematopoietic stem cell-specific antigen. In various embodiments, the antigen binding domain of antibodies that specifically bind an hematopoietic specific antigen are used to generate a CAR or TCR construct against the hematopoietic stem cell-specific antigen. Hematopoietic stem cell-specific antigens include, but are not limited to, c-kit (CD117), CD33, CD123 (IL-3Ralpha), CD133, CD135 CD105 and CD45.

Thus, in various embodiments, the construct encoding a cell-surface receptor comprises an antigen-binding domain of an immunoglobulin that specifically binds the hematopoietic stem cell-specific antigen. In various embodiments, the construct encodes amino acid sequences of a variable region, heavy chain (VH), a variable region, light chain (VL) or combinations thereof, from the immunoglobulin. Using antibodies known in the art, one of skill in the art can determine the appropriate combination of VH and/or VL regions from the immunoglobulin to include in the construct.

Examples of VH and VL regions for anti-c-Kit (CD117) antibodies include but are not limited to those described herein, the following patents: U.S. Pat. Nos. 5,919,911, 5,489,516, 8,552,157, and 8,436,150.

Examples of VH and VL regions for anti-CD123 (IL-3Ralpha) antibodies include but are not limited to those described herein, the following patents and patent applications: U.S. Pat. Nos. 8,163,279, 8,492,119, PCT/EP2016/051386, US20160297882, and US20140322212.

Examples of VH and VL regions for anti-CD135 (also known as Human Fms-like tyrosine kinase 3 receptor (FLT3), fetal liver kinase 2 (FLK-2) and stem cell tyrosine kinase 1 (STK-1)) antibodies include but are not limited to those described herein, the following patents: U.S. Pat. Nos. 8,071,099 and 9,023,996. One of skill in the art can identify VH and VL sequences, and the nucleic acid sequence encoding them, for a given antibody of known sequence. On of skill in the art can readily use such sequences to construct a CAR that targets a derived cell surface marker. Similar principles apply to the preparation of a modified T cell receptor (TCR).

An “antibody” also called “immunoglobulin” may be a natural or conventional antibody in which two heavy chains are linked to each other by disulfide bonds and each heavy chain is linked to a light chain by a disulfide bond. Each chain contains distinct sequence domains. The light chain includes two domains or regions, a variable domain (VL) and a constant domain (CL). The heavy chain includes four domains or regions, a variable domain (VH) and three constant domains (CH). The variable regions of both light (VL) and heavy (VH) chains determine binding recognition and specificity to the antigen. The specificity of the antibody resides in the structural complementarity between the antibody combining site and the antigenic determinant. As referred to herein, the terms “antibody” and “immunoglobulin” are used interchangeably and as used in the broadest sense and includes fully assembled antibodies, monoclonal antibodies (including human, humanized or chimeric antibodies), polyclonal antibodies, murine antibodies, human and mouse hybrids, multispecific antibodies (e.g., bispecific antibodies), and antibody fragments that can bind antigen (e.g., Fab′, F′(ab)2, Fv, single chain antibodies, diabodies), comprising complementarity determining regions (CDRs) of the foregoing as long as they exhibit the desired biological activity. The term “human antibody” as used herein, includes antibodies having variable and constant regions corresponding to human germline immunoglobulin sequences.

In various embodiments, a TCR used for targeting can be naturally occurring in the cell. In various other embodiments, CAR constructs are generated and used in other cells of the immune repertoire. In yet other embodiments, CAR constructs are generated for other species, including but not limited to mammals (such as humans, non-human primates, rodents, and domestic and game animals), primates (such as chimpanzees, cynomologous monkeys, spider monkeys, and macaques) and rodents (such as mice, rats, woodchucks, ferrets, rabbits and hamsters).

Various embodiments of the present invention also include the administration of an effective amount of one or more agents or treatments that are used in conventional conditioning. Regarding conventional conditioning treatment in the transplantation setting, the conditioning treatments may be determined by the skilled physician according to the particular needs of the patient. In various embodiments, high intensity conditioning may be used. In various other embodiments, reduced-intensity conditioning may be used. The T cells, NK cells or cytotoxic immune effector cells used in the methods and composition described herein allow for conditioning treatments that, under previously known transplant conditions, would not lead to engraftment. For example, use of T or NK cells administered at the time of transplantation promotes transplant engraftment under reduced conditioning treatments that would otherwise (e.g., without administration of such T or NK cells) not allow transplant engraftment. Thus, the conditioning used in the present invention is in a dose range such that with the administration of the cells as described herein, engraftment is achieved, while in the absence of such administration, engraftment would normally not be achieved.

In various embodiments, the cell compositions of the present invention are administered to a patient before, simultaneously with or following transplantation. In yet other embodiments, the conventional conditioning treatment can be administered before, simultaneously or following transplantation. Examples of conventional conditioning treatments include, but are not limited to, serotherapy, total body irradiation, chemotherapy (i.e., immune-depleting, myeoablative or stem-cell depleting), anti-lymphocyte globulin, anti-T-cell antibodies and/or immune-cell-depleting antibody treatment. In various other embodiments, conventional conditioning treatments can include, but are not limited to, fludarabine, external-beam radiation therapy (XRT), cyclophosphamide, OK.T3, CAMPATH, antithymocyte globulin (ATG), busulphan, dimethyl myleran, thiotepa, cyclosporin, azathioprine, methotrexate, mycophenolate, methyl prednisolone, prednisone, FK506, other immunoablative agents, anti-CD3 antibodies, cytoxin, etoposide, doxorubicin, vincristine, rapamycin, mycophenolic acid, steroids, FR901228, and irradiation.

In some embodiments, combinations of two or more conventional conditioning treatments can be administered. In other embodiments, the genetically modified cells (i.e., immune effector, T and/or NK cells) can be administered alone for conditioning or in combination with conventional conditioning treatments discussed herein. One of ordinary skill in the art would recognize the appropriate dosage and combination of treatments to be administered to the subject.

As discussed above, in various embodiments, an agent and/or drug or a treatment is administered to ablate the T cells, NK cells or cytotoxic immune effector cells administered to avoid proliferation and on-target toxicity against the donor stem cells. In some embodiments, a treatment is administered to kill the administered cells. For example, in various embodiments, the administered cells have been genetically modified to express an inducible gene, which when induced causes cell death. Examples of the inducible gene may include any one or more of the following: Herpes simplex type-1 virus (HSV1) thymidine kinase gene, a fusion between HSV1 thymidine kinase and zeocin-resistance gene, E. coli Cytosine Deaminase, E. coli Cytosine Deaminase fused to Uracil Phosphoribosyltransferase, S. cerevisiae Cytosine Deaminase, S. cerevisiae Uracil Phosphoribosyl Transferase, Equine herpes virus 4 (EHV4) thymidine kinase, Herpes simplex virus 1 (HSV1) thymidine kinase, HSV1 thymidine kinase fused to Zeocin-resistance gene, E. coli thymidine kinase fused to thymidylate kinase, E. coli Uracil Phosphoribosyltransferase, and dimerizable, modified human caspase 9 fused to a human FK506. In alternative embodiments, the T or NK cells are rendered dependent upon the continued presence of an agent, such that removal, rather than administration of the agent results in cell death.

In various embodiments, the administered cells have been genetically modified with a construct comprising a truncated EGFR with an intact cetuximab binding site, for cell ablation. In other embodiments, cetuximab is administered which results in in vivo cell ablation.

The T cells, NK cells or cytotoxic immune effector cells of the present invention can be administered before, during or after a cell/organ transplant. In various embodiments, the cells are administered at the same time as the transplant. In other embodiments, the cells are administered within 24 hours of the transplant. In yet another embodiment, the cells are administered within less than 1, 2, 3, 4, 5, 6, 7, 8, 9, or 10 days of the transplant. As would be recognized by the skilled artisan, the number of T cells, NK cells or cytotoxic immune effector cells administered to the transplant patient will vary depending on the condition of the patient and thus can be determined by a qualified physician. In various embodiments, the number of T cells, NK cells or cytotoxic immune effector cells administered may be between 1×10⁶ to 1×10¹¹ cells/kg (body weight). For example, the number of cells administered may be about 1×10⁶ to 1×10⁸ or 1×10⁸ to 1×10¹¹ cells/kg. In various other embodiments, the number of T cells, NK cells or cytotoxic immune effector cells administered may vary depending on the conditioning treatment administered.

In various embodiments, the T cells, NK cells or cytotoxic immune effector cells can be administered in multiple infusions. In various embodiments, the cells can be administered systemically. In some embodiments, the population of cells can be administered locally. In various other embodiments, the cells can be administered intravenously, intraarterially, subcutaneously, or intraosseously. The administration of the cells can be carried out in any convenient manner, including by injection, transfusion, implantation or transplantation.

In various embodiments of the present invention, donor stem cells are administered to the subject. In various other embodiments, autologous stem cells are administered to the subject. In various embodiments, the donor (allogenic) and/or autologous stem cells are administered to the subject using methods known in the art. In other embodiments, the donor (allogenic) and/or autologous stem cells are administered to the subject using methods described herein for administering T cells, NK cells or cytotoxic immune effector cells.

### Compositions

The cell compositions described herein are useful, for example, in a variety of applications including, but not limited to, promoting engraftment and therapeutic treatment for malignant and non-malignant diseases. The methods of use can be in vitro, ex vivo, or in vivo methods. In certain embodiments, the cell composition is a population of genetically modified T cells, NK cells or cytotoxic immune effector cells.

In various embodiments, the pharmaceutical compositions according to the invention can be formulated for delivery via any route of administration. “Route of administration” may refer to any administration pathway known in the art, including but not limited to parenteral.

“Parenteral” refers to a route of administration that is generally associated with injection, including infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular or subcutaneous. Via the parenteral route, the compositions can be in the form of solutions or suspensions for infusion or for injection.

In various embodiments, the cell composition can be administered intravenously by injection or by gradual infusion over time. Given an appropriate formulation for a given route, for example, the cell composition useful in the methods described herein can be administered intravenously, intraarterially, subcutaneously, or intraosseously, and can be delivered by peristaltic means, if desired, or by other means known by those skilled in the art.

The cell compositions according to the invention can also contain a pharmaceutically acceptable carrier. “Pharmaceutically acceptable carrier” as used herein refers to a pharmaceutically acceptable material, composition, or vehicle that assists in establishing or maintaining the cell composition in a form for administration. For example, the carrier may be a liquid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

In various embodiments, the present invention provides pharmaceutical compositions including a pharmaceutically acceptable excipient along with a therapeutically effective amount of the cell composition. “Pharmaceutically acceptable excipient” means an excipient that is useful in preparing the cell composition that is generally safe, non-toxic, and desirable, and includes excipients that are acceptable for veterinary use as well as for human pharmaceutical use. The active ingredient, e.g., cells, can be mixed with excipients which are pharmaceutically acceptable and compatible with the active ingredient and in amounts suitable for use in the therapeutic methods described herein. To the extent compatible with the cells, a cell composition as described herein can include pharmaceutically acceptable salts. Pharmaceutically acceptable salts include the acid addition salts formed with inorganic acids such as, for example, hydrochloric or phosphoric acids, organic acids, for example, acetic, tartaric or mandelic, salts formed from inorganic bases such as, for example, sodium, potassium, ammonium, calcium or ferric hydroxides, and salts formed from organic bases such as isopropylamine, trimethylamine, 2-ethylamino ethanol, histidine, procaine and the like. Physiologically tolerable carriers are well known in the art. The amount of an active agent used in the invention that will be effective will depend on the nature of the disorder or condition, and can be determined by one of skill in the art with standard clinical techniques.

The cell compositions as described herein can be administered either alone, or as a cell composition in combination with diluents and/or with other components such as cytokines or cell populations. The cell composition can comprise a combination of one or more pharmaceutically or physiologically acceptable carriers, diluents or excipients, discussed above.

The cell compositions according to the invention can be delivered in an “effective amount”. The precise therapeutically effective amount is that amount of the composition that will yield the most effective results in terms of efficacy of engraftment and/or treatment in a given subject. This amount will vary depending upon a variety of factors, including but not limited to the characteristics of the cell composition (including activity and receptor function), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the art will be able to determine a therapeutically effective amount through routine experimentation, for instance, by monitoring a subject's response to administration of the cell composition and adjusting the dosage or administration regimen, accordingly.

Typical dosages of an effective cell composition can be as indicated to the skilled artisan by the in vitro responses or responses in animal models. Such dosages typically can be reduced by up to about one order of magnitude in concentration or amount without losing the relevant biological activity. Thus, the actual dosage will depend upon the judgment of the physician, the condition of the patient, and the effectiveness of the therapeutic method based, for example, on the in vitro responsiveness of the relevant primary cultured cells, cell lines or histocultured tissue sample, such as biological samples obtained, or the responses observed in the appropriate animal models. As discussed above, in some embodiments, the number of T cells, NK cells or cytotoxic immune effector cells administered can be between 1×10⁶ to 1×10¹¹ cells/kg to the subject. In some other embodiments, the number of cells administered can be greater than 1×10¹¹ cells/kg. In various other embodiments, the cells may be autologous or allogeneic to the subject undergoing transplant.

For the treatment of malignant and non-malignant disease, the appropriate dosage of cell compositions of the present invention depends on the type of disease to be treated, the severity and course of the disease, the responsiveness of the disease, whether the cell composition is administered for therapeutic or preventative purposes, previous therapy, and patient's clinical history. The dosage can also be adjusted by the individual physician in the event of any complication and at the discretion of the treating physician. The administering physician can determine optimum dosages, dosing methodologies and repetition rates.

While short duration is preferred to condition a transplant recipient before the administering a transplant, the cell compositions can be administered one time or over a series of administrations. The cell compositions of the present invention can be administered in multiple, sequential dosages as determined by a clinician.

### EXAMPLES

The following examples are not intended to limit the scope of the claims to the invention, but are rather intended to be exemplary of certain embodiments. Any variations in the exemplified methods which occur to the skilled artisan are intended to fall within the scope of the present invention.

### Example 1

As killer cells, primary autologous or allogeneic cytotoxic T-cells, NK-cells or stable T or NK-cell lines endowed with killer machinery can be used, for example. Naturally occurring T- or NK-cell species could be selected for targeting, or cells could be genetically modified for instance by overexpressing a chimeric antigen receptor (CAR) or T-cell receptor (TCR) against surface antigens expressed on hematopoietic stem cells, which serve as targets. Any hematopoietic-specific surface antigen would be suitable, as a target, in order to reduce or avoid on-target, off-stem cell toxicity. Cells would be administered systemically, for instance, intravenously, intraarterially, subcutaneously or intraosseously. Conditioning could be with targeted killer cells alone or in combination with a serotherapy and/or a reduced-intensity conventional conditioning, for instance with reduced-dose total body irradiation or reduced-dose chemotherapy, combined for instance with anti-lymphocyte globulin (ATG). The killer cells should not persist in relevant numbers in vivo, either due to spontaneous death, by high-dose ex vivo irradiation or induced death by means of, for instance, drug-inducible mediators of apoptosis such as thymidine kinase, and should not be able to proliferate in vivo for extended periods of time (beyond the time of transplantation of the stem cell graft) so as to avoid on-target toxicity against the donor cells.

### Example 2

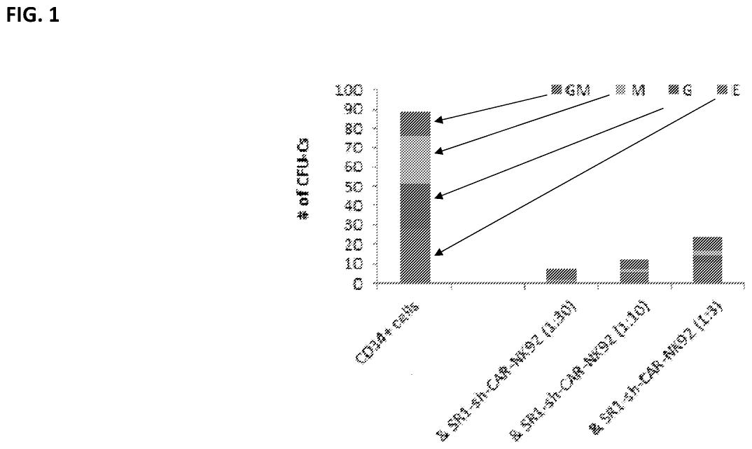

Proof-of-principle data was generated by cloning a conventional second-generation chimeric antigen receptor (CAR) against the hematopoietic-specific, stem-cell enriching antigen “c-kit”, based on an anti-human-c-kit antibody generated referred to as, SR1. The CAR was cloned into a lentiviral vector which was used to transduce NK92 cells, an IL-2 dependent continuously growing NK cell line. CAR-expressing NK92 cells were enriched by FACS sorting using a co-expressed selectable marker in this instance, an EGFR cassette, but others will work similarly. To assess killing of hematopoietic stem cells in vitro, SR1-NK92 cells or parental NK92 cells were co-incubated with primary bone marrow consisting of a mix of cells including a minority population of clonogenic cells, subsequently plated in clonogenic assays (CFU-C assays). Clonogenic cells were enumerated after 14 days. Bone marrow not incubated with NK92 cells as well as bone marrow incubated with parental NK92 cells served as controls for specific killing activity. In the bone marrow sample, c-kit+ cells (target cells) were enumerated, and NK92 or SR1-NK92 cells were added at ratios of 3:1, 10:1 or 30:1 c-kit+ cells. In FIG. 1, SR1-NK92 cells demonstrate a dose-dependent killing activity against clonogenic cells of >90% at a 30:1 effector:target ratio.

Without being bound to any particular theory, based on these data and on published data by ourselves on Her2Neu-CAR-modified NK92 cell (Schönfeld et al. Mol Ther. 2015 February; 23:330-8), it is specifically contemplated that, when administered systemically, SR1-NK92 cells can ablate hematopoietic stem cells in vivo and thus allow engraftment of a stem cell graft, irrespective of the genetic relationship between donor and recipient. For successful engraftment, killer cells can be administered by themselves, or combined with additional modalities such as, reduced intensity radio-/chemotherapy conditioning and antibody-mediated immune cell depletion.

The same principle can be applied to any other primary cell or cell line, genetically modified with a CAR, a TCR or any other construct conveying specific anti-stem cell immune recognition, or selected from the natural immune cell repertoire.

### Example 3

### CFU-C Cytotoxicity Assay

Unmanipulated healthy donor bone marrow (i.e. mix of mature and immature blood cells representative of marrow cell content) was co-incubated with NK92 cells redirected against the antigen c-kit (SR1-sh NK92) or parental NK92 cells at three different effector target ratios (30:1, 10:1 & 3:1). Effector target ratio was calculated according to the number of c-kit expressing cells in the cell mix as measured by flow cytometric analysis.

300 CD34+ stem cells were incubated with the indicated number of effector cells in 300 μL assay medium (X-Vivo 10, supplemented with 5% human plasma, 500 IU/ml IL-2 and 10 ng/ml hTPO) for 20 hours in a 96-well-plate. Thereafter, the cell mix was added to 2 ml of cytokine-replete methylcellulose medium (StemMACS HSC-CFU with Epo, Miltenyi Biotec) and divided onto two 35-mm plates. After 10-14 days under normal culture conditions (saturated humidity, 5% CO2, 37° C.) CFU-Cs were enumerated. Untreated stem cells and stem cells treated with parental NK92 cells were included as controls. For experiments #4 and #5, a mix of parental and CAR-modified NK92 cells was generated, containing 30+0, 0+30, 20+10 or 27+3 parental and CAR-modified NK92 cells, respectively, for each CD34+ cell, to control for (killer) cell density which may affect clonogenic growth simply by crowding and competition for nutrients. In these cases, the “NK92-WT 1:30” (equivalent to 30+0 parental and CAR-modified NK92 cells) is the relevant control, in all other experiments the delta between identical concentrations of parental and CAR-modified NK92 defines the specific lysis.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

1. A method of promoting engraftment of a hematopoietic stem cell (HSC) transplant, the method comprising:
administering, to a subject in need of a HSC transplant, a composition comprising a population of T cells, NK cells or cytotoxic immune effector cells comprising a cell-surface receptor for a hematopoietic stem cell-specific antigen, the amount effective to ablate the stem cells with the hematopoietic stem cell-specific antigen in the subject.

2. The method of claim 1, wherein the T or NK cells are cytotoxic T or NK cells.

3. The method of claim 1, wherein the population of T cells, NK cells or cytotoxic immune effector cells is autologous to the subject.

4. The method of claim 1, wherein the population of T cells, NK cells or cytotoxic immune effector cells is allogeneic to the subject.

5. The method of claim 1, wherein the population of T cells, NK cells or cytotoxic immune effector cells comprise a heterologous nucleic acid encoding the cell-surface receptor.

6. The method of claim 1, wherein the cell surface receptor comprises a chimeric antigen receptor (CAR).

7. The method of claim 1, wherein the cell surface receptor comprises a T cell receptor (TCR).

8. The method of claim 1, wherein the cell surface receptor specifically binds a hematopoietic stem cell-specific antigen selected from the group consisting of c-kit (CD117), CD33, CD123 (IL-3Ralpha), CD133, CD135, CD105 and CD45.

9. The method of claim 1, wherein the population of T cells, NK cells or cytotoxic immune effector cells is administered systemically.

10. The method of claim 9, wherein the population of T cells, NK cells or cytotoxic immune effector cells is administered intravenously, intraarterially, subcutaneously, or intraosseously.

11. The method of claim 1, further comprising administering an effective amount of one or more agents or treatments that are used in conventional conditioning.

12. The method of claim 11, wherein the agent or treatment is selected from serotherapy, total body irradiation, chemotherapy (i.e., immune-depleting, myeoablative or stem-cell depleting), anti-lymphocyte globulin, anti-T-cell antibodies and/or immune-cell-depleting antibody treatment.

13. The method of claim 1, further comprising administering donor stem cells to the subject.

14. The method of claim 13, further comprising ablating the T cells, NK cells or cytotoxic immune effector cells to avoid proliferation and on-target toxicity against the donor stem cells by administering an agent and/or drug or a treatment in an amount sufficient to kill the administered population of T cells, NK cells or cytotoxic immune effector cells.

15. The method of claim 1, wherein the population of T cells, NK cells or cytotoxic immune effector cells comprises one or more heterologous nucleic acids encoding one or more polypeptides that induces cell death in the administered population of T cells, NK cells or cytotoxic immune effector cells using a selected inducing agent and/or drug.

16. The method of claim 14, wherein administering the agent, drug and/or treatment induces apoptosis of the administered population of T cells, NK cells or cytotoxic immune effector cells.

17. The method of claim 1, wherein the administered population of T cells, NK cells or cytotoxic immune effector cells eradicate the subject's hematopoietic stem cells.

18. A method of killing hematopoietic stem cells in a subject in need of an hematopoietic stem cell transplant, the method comprising:
administering, to a subject in need of hematopoietic stem cell transplantation, a composition comprising a population of T cells, NK cells or cytotoxic immune effector cells comprising a cell-surface receptor for an hematopoietic stem cell-specific antigen.

19. The method of claim 18, wherein the population of T cells, NK cells or cytotoxic immune effector cells is autologous to the subject.

20. The method of claim 18, wherein the population of T cells, NK cells or cytotoxic immune effector cells is allogeneic to the subject.

21. The method of claim 18, wherein the population of T cells, NK cells or cytotoxic immune effector cells comprise a heterologous nucleic acid and/or protein encoding the cell-surface receptor.

22. The method of claim 18, wherein the cell surface receptor comprises a chimeric antigen receptor (CAR).

23. The method of claim 18, wherein the cell surface receptor comprises a T cell receptor (TCR).

24. The method of claim 18, wherein the cell surface receptor specifically binds a hematopoietic stem cell-specific antigen selected from the group consisting of c-kit (CD117), CD33, CD123 (IL-3Ralpha), CD133, CD135, CD105 and CD45.

25. The method of claim 18, wherein the population of T cells, NK cells or cytotoxic immune effector cells is administered systemically.

26. The method of claim 25, wherein the population of T cells, NK cells or cytotoxic immune effector cells is administered intravenously, intraarterially, subcutaneously, or intraosseously.

27. The method of claim 18, further comprising administering an effective amount of one or more agents or treatments that kills hematopoietic stem cells.

28. The method of claim 27, wherein the agent or treatment is selected from total body irradiation, anti-lymphocyte globulin, and immune-cell-depleting antibody treatment.

29. The method of claim 18, further comprising administering donor hematopoietic stem cells to the subject.

30. The method of claim 18, further comprising ablating the T cells, NK cells or cytotoxic immune effector cells to avoid proliferation and on-target toxicity against the donor hematopoietic stem cells by administering an agent and/or drug or a treatment in an amount sufficient to kill the administered population of T cells, NK cells or cytotoxic immune effector cells.

31. The method of claim 18, wherein the population of T cells, NK cells or cytotoxic immune effector cells comprises one or more heterologous nucleic acids encoding one or more polypeptides that induces death in the administered population of T cells, NK cells or cytotoxic immune effector cells using a selected inducing agent and/or drug.

32. The method of claim 30, wherein administering the agent, drug and/or treatment induces apoptosis of the administered population of T cells, NK cells or cytotoxic immune effector cells.

33. The method of claim 18, wherein the administered population of T cells, NK cells or cytotoxic immune effector cells eradicate the subject's hematopoietic stem cells.

34. A composition comprising T cells, NK cells or cytotoxic immune effector cells genetically modified to encode a cell-surface receptor for a hematopoietic stem cell-specific antigen.

35. The composition of claim 34, wherein the cell surface receptor for a hematopoietic stem cell-specific antigen specifically binds a hematopoietic stem cell-specific antigen selected from the group consisting of c-kit (CD117), CD33, CD123 (IL-3Ralpha), CD133, CD135, CD105 and CD45.

36. The composition of claim 35, wherein the genetic modification encoding the hematopoietic cell-surface receptor encodes an antigen-binding domain of an immunoglobulin that specifically binds the hematopoietic stem cell-specific antigen.

37. The composition of claim 35, wherein the construct encoding a cell-surface receptor comprises an antigen-binding domain of an immunoglobulin that specifically binds the hematopoietic stem cell-specific antigen.

38. The method of claim 34, wherein the genetically modified T or NK cells are cytotoxic T or NK cells.
